(19) United States
(12) Patent Application Publication
(10) Pub. No.: US 2017/0281464 A1
(43) Pub. Date: Oct. 5, 2017

(54) Automated Heart-Abdomen External Masseur

(71) Applicant: Mojtaba Hadizadeh, Alborz Industrial city (IR)

(72) Inventor: Mojtaba Hadizadeh, Alborz Industrial city (IR)

(21) Appl. No.: 15/089,602

(22) Filed: Apr. 4, 2016

(51) Int. Cl. A61H 31/00; A61N 1/39; A61B 5/1455; A61B 5/00; A61B 5/044; A61B 5/0205

(52) U.S. Cl. CPC A61H 31/006; A61H 31/005; A61B 5/044; A61B 5/02055; A61B 5/14551; A61B 5/6814; A61N 1/3993; A61H 2201/1207; A61H 2201/5082; A61H 2201/5089

(57) ABSTRACT

A Cardio Pulmonary Resuscitation (CPR) device, consisting of two distinct masseurs for chest and abdomen and a monitoring system for displaying patient's vital symptoms. The chest masseur works in controllable depth and speed, while the abdominal masseur applies pressure on abdominal aorta and Inferior vena cava in a contradictory rhythm with the chest masseur rhythm. This action causes more effective massaging by preventing overflow of blood to non-vital organs and accelerating return of blood to heart.

### FIELD OF INVENTION

This invention is an electronic-mechanical device that fulfills Cardio Pulmonary Resuscitation (CPR) automatically with its wide lateral features.

### BACKGROUND OF THE INVENTION

In common methods, in order to help a patient during cardiac arrest, the rescuer puts one hand on the top of the other hand and together on the patient's chest and while his arms are in a straight position, presses the middle part of patient's chest (sternum). Taking into account that the rescuer has to consume extra energy during this swift and non-stop action, the results are noticeably depending on rescuer's capabilities. Among the disadvantages of traditional CPR; exhaustion of rescuer, quality deduction of the CPR after a certain lapse of time, delay in other necessary activities like opening the air canals, medicine injection and use of defibrillator; could be mentioned. According to statistical studies, more than 50% of survivors have brain damage.

In order to carry out the resuscitation effectively by machines, many devices have been invented for cardiac massage. First generation of such devices were completely designed on mechanical basis and applied manually. In further generations of invented devices, a combination of electronic and mechanical science was utilized to operate device automatically.

Invented devices incorporated here by reference in U.S. Pat. No. 5,743,864; U.S. Pat. No. 7,775,996 B2; U.S. Pat. No. 8,092,404 B2; U.S. Pat. No. 4,424,806; U.S. Pat. No. 6,398,745 B1 are some of such apparatuses created for cardiac massage.

The invented devices usually consist of mechanical, electrical and hydraulic or pneumatic elements; therefore, they are heavy in comparison with their efficiency.

High price, time consumption and difficult starting, disorderliness around the patient's bed in many of invented models are some of the disadvantages of the prior art. Also lack of auxiliary features to survive patient like defibrillation and monitoring of vital symptoms, need to remove all of patient's clothing and the necessity to cover the entire surface of the patient's chest; full compression instead of partial effective compression, are the other disadvantages observed in some of the invented models. The risk of brain damage still exists even in the best and most efficient ones.

### SUMMARY OF THE INVENTION

The invented device; a combination of compression on both chest and abdomen automatically; raises the probability of longer life of patient without serious damages on the brain. Also by utilizing a vital symptom display, central control system, defibrillator, quick starting to save time and smaller size to increase maneuverability of medical team and lower weight to handle easily; have solved many of the aforementioned problems in the prior art, therefore CPR process can be done more efficiently.

Alternative method was invented to minimize brain damages by applying extra compression on abdominal aortic and inferior vena cava. When the rescuer is pressing the chest in this method, he/she is applying another pressure on the patient's abdomen or in case of more than one rescuer, whenever one is working on the chest the other compresses the abdomen simultaneously. This action either prevents flowing blood towards lower organs and circulating it to non-vital organs by compressing abdominal aortic. Compressing Inferior vena cava leads to an increase of blood velocity and volume that returns to the heart.

In the first step after diagnosing cardiac arrest and starting CPR process, the rescuer places chest masseur on the patient's body and run it at 100 times per min, with a 2″ depth (minimum requirement).

The applied compressions moves patient's chest back and forth (inside and out) to facilitate chest returning to its normal condition. The display monitor shows vital symptoms of the patient on a touch-screen; such as but not limited to cardiogram, heartbeat, pulsation, blood oxygen contents, body temperature and blood pressure. The rescuer can easily control the functions with the touch monitor. Moreover, against many of invented devices, this invention is able to recognize and analyze heart rhythm and the needed shock frequency, and therefore if necessary intelligently applying the proper shock.

After this step, the rescuer adds abdominal compression in sequence to process, which applies pressure on the abdominal aorta and Inferior vena cava at a contradictory rhythm in comparison with chest compression compartment. Compressing abdominal aorta prevents overflow of blood to lower limbs and improves blood flow toward heart and brain. This phenomena leads to survival of the patient in longer periods. Compressing inferior vena cava also accelerate backing blood to heart, so damages are kept minimized due to inadequacy of blood in the heart and the brain during cardiac arrest.

It is worthy to mention that abdominal and chest masseur compartments are quite separate and rescuer can ignore the abdominal one if deem, advisable. Both batteries and network electricity can supply the power of the device, therefore it can be used in ambulances, emergency services, and surgery rooms.

### BRIEF DESCRIPTION OF THE DRAWINGS

FIG. 1 shows the tridimensional view of the invented automatic heart-abdomen massage device.

FIG. 2 represents the tridimensional view of how the invented device located on the patient's body.

FIG. 3 is the front view of the interior elements of invented chest masseur frame.

FIG. 4 shows how to locate elements of the invention on the patient body.

FIG. 5 shows the tridimensional view of the abdomen masseur of the invention, separately.

FIG. 6 shows the top view of internal elements placed in the frame of the invented abdomen masseur.

FIG. 7 is a schematic illustration of the power supply of the invention.

FIG. 8 is a scheme to shows the connection of different parts of the device to the central control unit.

### DETAILED DESCRIPTION OF THE INVENTION

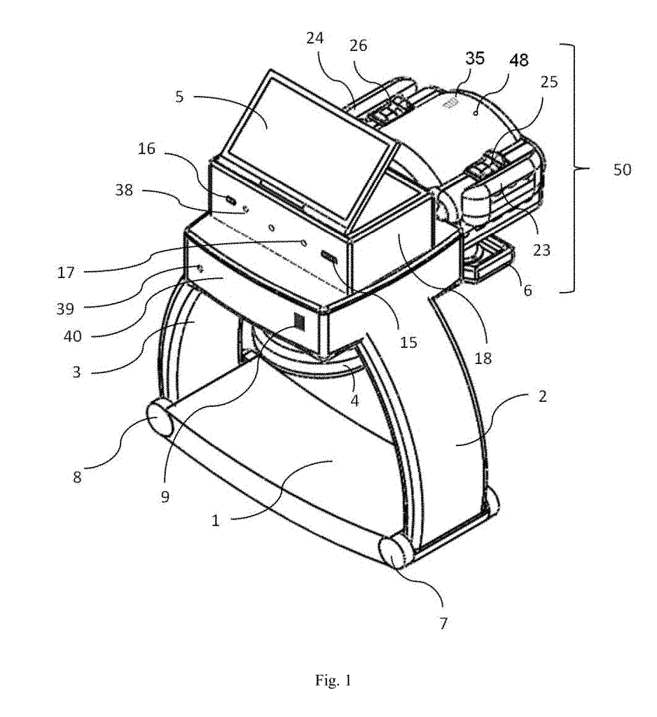

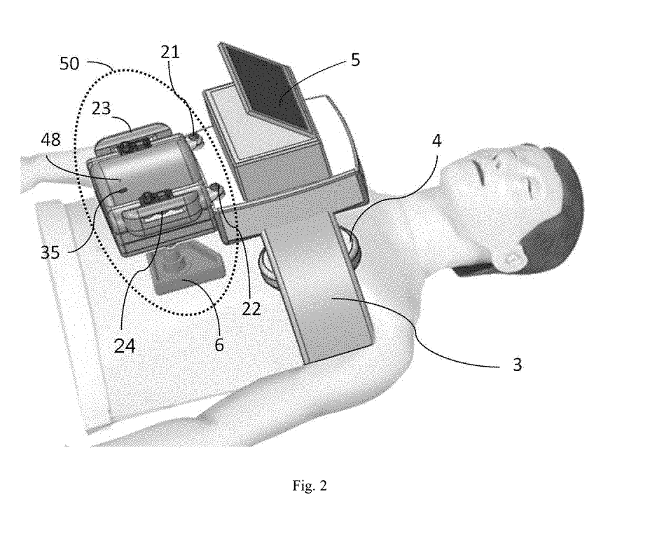

According to FIG. 1 chest masseur is equipped with plate (1) placed under the patient's back. All main parts of apparatus assembled on two arms (2 and 3). These parts are made of non-flexible carbon fiber material. Flexible pad (4) that acts as the chest masseur. Vital symptoms can be observed via monitor (5) and abdomen massage fulfills by pressing pad (6). After a cardiac arrest, the rescuer place plate (1) under patient's back and shoulder as arms (2 and 3) located between hands and body of the patient as shown in FIG. 2 and pad (4) place on the middle of the chest. It should be said in FIG. 1, width of plate (1) can be adjusted manually to patient's body size by the rescuer by sliding joints (7 and 8).

The rescuer power up the device by an on/off switch (9) and run the device by finger touch screen (5) which is made with LED technology. In the case of low battery, the rescuer can connect network electricity to inlet (39).

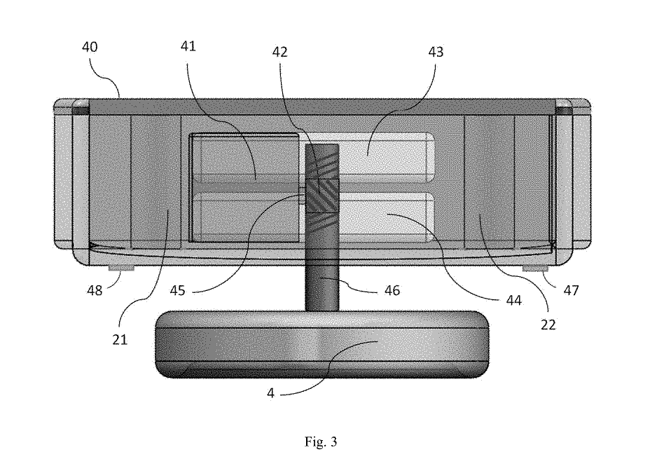

FIG. 3 shows front view of the inside of frame (40) of the chest masseur. Driver gear (42) has been coupled to shaft (45) of motor (41). This gear (42) together with teeth on massage shaft (46) form a set of rack and pinion mechanism. Therefore rotational movement of gear (42) causes up and down movement of massage shaft (46). Consequently, vertical movement of massage shaft (46) causes vertical movement of flexible chest masseur pad (4) which is attached to massage shaft (46). Two ultrasonic sensors (47 and 48) measure the distance between pad (4) and the patient chest. Flexible suction pad/chest masseur (4) moves down just equal to the distance measured, and are placed on the patient's chest.

After settlement of pad/chest masseur (4) on the chest, continuous movement of pad (4) causes compression and decompression on the patient's chest and consequently massaging the chest at minimum 100 times per min (at least 100 times per min). With a minimum 2″ depth occurs. These quantities can be seen and controlled on monitor (5) (shown in FIG. 1) by the rescuer. This kind of massage forces the patient's chest inside and out to accelerate returning chest to its normal position.

Batteries 43 and 44 are located on the backside of motor (41) and are made up of lithium Ion and lithium polymer. Their nominal voltages are 24 and 12 volts, respectively; however they are not limited to these numbers and can be changed as needed.

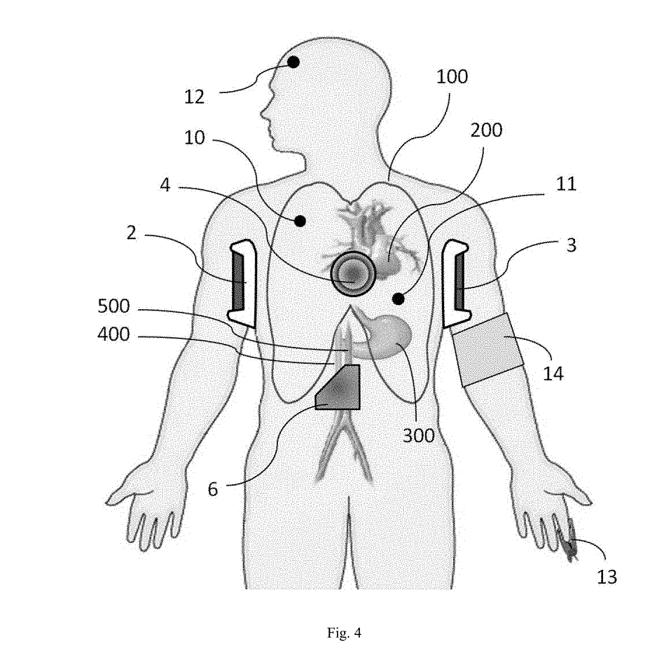

After starting massage job, As shown in FIG. 4, the rescuer immediately connects sphygmomanometers cuff (14) to the left arm of the patient, defibrillator and ECG lids to sternum (10) and apex (11), body temperature sensors lid (12) on the patient's forehead and pulse oximetry probe (13) to his/her forefinger. Furthermore, pad/chest masseur (4) is located on chest (100) and is massaging the heart (200).

Pad/abdomen masseur (6) which is placed on chest (100) and stomach (300), on inferior vena cava (400) and abdominal aorta (500), can be seen in this figure. Cables connecting elements are shown in FIG. 1. Pulse oximetry probe is jointed to inlet (16) and the amount of SPO2 and pulsation are observed on monitor (5). Sphygmomanometers cuff (14) is connected to inlet (17) and blood pressure quantity can be recorded, if necessary. Temperature sensor's cable is connected to terminal (38) and defibrillator and ECG lids are connected to terminal (15), so the cardiogram and heartbeat can be observed on monitor (5).

The central control unit installed in part (18) compares patient's cardiogram to pre-defined waves necessary for shocking, and then warns rescuers by monitor display (5) about the risks. Now, the rescuer is able to assign proper sort and amount of shock by monitor (5). This device automatically discharges electrical shocks through lids (10) and (11) after a few seconds. Then by analyzing ECG wave, if necessary the system continues massaging automatically if necessary. Otherwise, remains in standby mode, until operator commands.

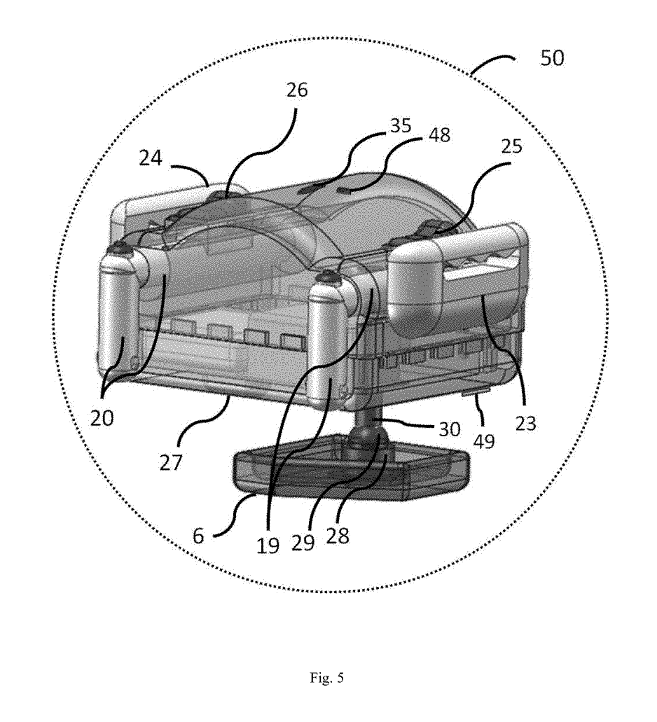

Other essential part of abdominal masseur package (50) can be seen in FIG. 5. In this figure, in order to see the interior elements, frame (27) has been drawn transparently. This compartment of the device performs as abdominal masseur and after turning on the chest masseur, will be connected thereto. To apply abdominal massage, the rescuer places vertical section of L shaped arms (19 and 20), inside two spline holes ((21 and 22) as shown in FIG. 2) and locks them. Horizontal sections of L shaped arms (19 and 20) are also fitted in two holes on frame (27). Therefore the rescuer holds handles, pushes the bottoms (25 and 26) and by releasing spline and movement of abdominal arms (19 and 20), can adjust the distance between the chest and abdomen parts. As it can be seen in FIG. 4, the rescuer adjusts distance to locate upper face of pad (6) place below chest (100). Shape of pad (6) is specially designed to minimize pressure on patient stomach and intestines, and prevents the risk of aspiration of stomach (300) content to lung.

The designer also considered the risk of extra pressure to the chest bones and connects pad (6) by part (28) to the spiral joint (29), as it can be seen in FIG. 5. If pad (6) is placed on the chest, joint (29) will prevent inserting too much force on the chest's bones by inclining pad (6).

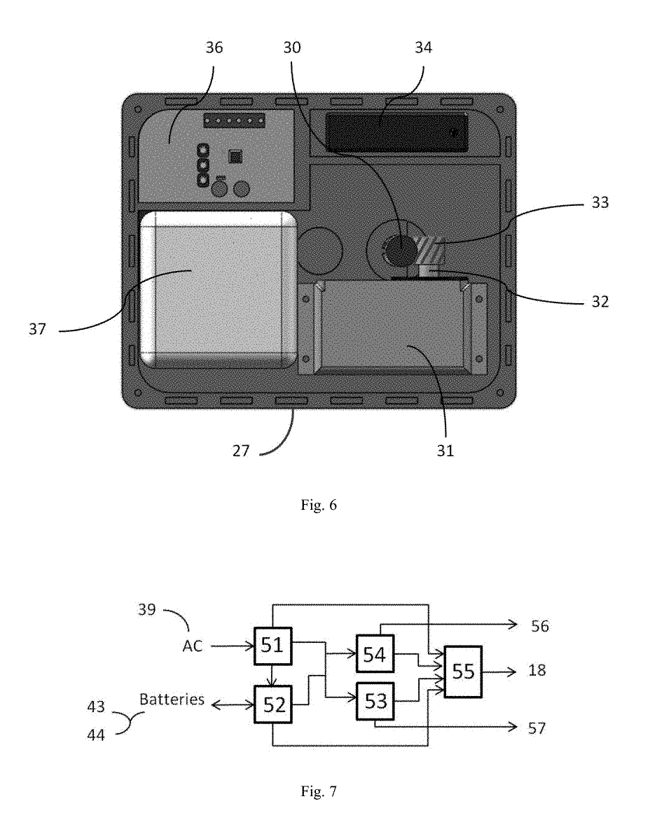

The rescuer turns on the abdominal masseur by switch (35). Abdominal masseur has wireless connection to the chest masseur and is controlled by monitor ((5) in FIG. 1). Motor ((31) in FIG. 6) is placed in central section of abdominal masseur and as will be described in FIG. 6, pressing arm (30) is moved down straightly after receiving data from ultra-sonic receiver (49). So that pad (6) is set in tangent position with the patient's abdomen. Then speed and depth are adjusted to the same numbers as the chest device, but with a contradictory rhythm. A certain pressure is applied on inferior vena cava ((400) in FIG. 4) and abdominal aorta ((500) in FIG. 4). This action compresses abdomen about half an inch.

FIG. 6 shows inside of the abdomen masseur's frame (27) from the top view. As it can be seen, shaft (32) of motor (31) is coupled to gear (33). This gear together with the teeth on arm (30), form a set of rack and pinion mechanism like on the chest masseur one; so when motor (31) runs, gear (33) rotates and arm (30) moves vertically, consequently. Lithium Ion 24 volts battery (37), XBEE module (34) and processor board (36) also are shown in this figure as well.

Whenever the system runs, the device starts sending & receiving data to central control unit (18) by XBEE module (34) and data is transferred to processor (36) and decision can be made for motor (31). During the operation, LED (48) (can be seen in FIG. 5) is mounted on the device, and is blinking and when linking process is done; the LED will remain, turned on.

Both batteries and network electricity can supply the device. According to FIG. 7 in a network mode, DC/AC converter (51) changes the electrical current that enters via inlet (39), to DC with a lower tension. In addition, this system charges the batteries (43 and 44 in FIG. 3) by a battery control circuit (52). The outputs of battery control circuit (52) and DC/AC convertor (51) are used to supply 12 and 24 volts DC/DC convertors (53 and 54).

These convertors transform DC current to a regulated and stable current, which will be apply to some of control unit (18) elements and send to voltage detect circuit (55). This circuit senses an output of other circuits, changes analogues signal to digital mode, and transfers them to the central control unit (18) for more processing. Therefore energizing the system and charging of the batteries occurs in a same time. If the AC current is disconnected, the output of converter (51) will be disconnected to converters (53 and 54).

Therefore, the control circuit (52) uses the battery energy as power supply. Current from converter (51) and control circuit (52) also supply circuit (55), directly. So four inputs of circuit (55), by considering the necessary type and voltage amount, supplies power to elements of central control unit (18). 12 and 24 volt output of (56 and 57) are provided by converters (53 and 54) directly and therefore other elements of the device like motor and defibrillator will start operating. Supply of abdominal section is also provided by its battery (37) (in FIG. 6) independently.

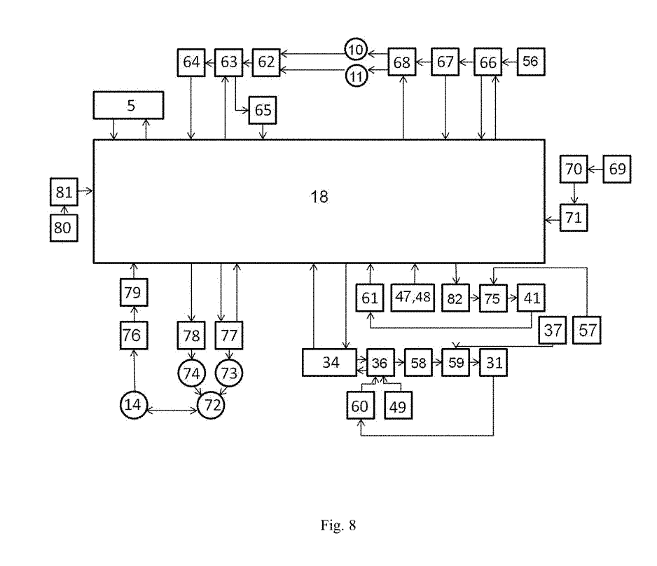

In FIG. 8, the central control unit (18) can be observed. This section like many other control systems is equipped with CPU consisting of logical control circuit, RAM, ROM and watchdog. When the device starts working, the rescuer is able to command and control the functions by touch screen (5). Central control unit (18) from finger touch screen (5) receives the commands. Signals from the monitor must be transmitted to the chest and abdomen masseur's motors (31 and 41) for movement controls. Signals from ultra-sonic sensors (47 and 48) of abdominal masseur are sent to central adjust section (18), then to PID controller (82) to control the speed and rotation direction of motor (41), and then to motor driver (75). Motor driver (75) controls motor (41) performance by PWM method.

During the abdomen massage process, data that is entered by monitor (5), and chest and abdominal masseur's data, will be exchanged between central control unit (18) and processor board (36) by XBEE module (34), wirelessly. PID controller (58) and motor's driver (59) control the motor (31) by output signal of Processor board (36). (60 and 61) also are F/V converters that send feedbacks to central control unit (18) in order to lessen the errors of controlling. Power supply of motors (31) and (41) is provided by output current (57) of convertor (53) in FIG. 7 and battery (37) via their drivers (75) and (59), respectively.

There is also a schematic illustration of defibrillator and ECG circuit in this figure. Output signals of lids (10) and (11) are sent to ECG signal input circuit (62). This circuit isolates entering high voltage surges of defibrillator from monitoring section and selects certain frequencies as a filter to clarify cardiogram. The signals, then, are entered to ECG signal processing circuit (63). This electrical circuit is responsible for amplifying cardio signals and distinguishes them from noises. After that, digital signals from A/D converter (64) are sent to central control unit (18). In addition, the output of signal from circuit (63) has been sensed by pace detect circuit (65) to count the heartbeat from cardiogram and send its data to unit (18).

In order to apply electric shocks, high voltage charger (66) should be energized by output (57) that is a 12V current (as to be seen in FIG. 7). High voltage charger (66) charges high voltage capacitor (67) and is connected to unit (18) to set the exact time of applying shock. High voltage capacitor (67) also is connected to unit (18) to transfer information about amount of charged and required voltages. Switching/isolation circuit (68) does not allow discharging of energy while charging is in process. In this situation, output of lids (10) and (11) are sent to circuit (62) and ECG signals will continue to be taken. When high voltage capacitor (67) is charged to a certain amount that was pre-selected on screen (5), circuit (68) turns to switching mode to discharge high voltage capacitor (67) via lids (10) and (11) for applying shock to the patient.

When the rescuer connects pulse oximetry probe to the patient's finger ((13) in FIG. 3), sensor (69) glows Infrared and spectrum to the patient's blood and uses reflected light to produce SPO2 signals. The signals enter in the SPO2 signal processing circuit (70), and are converted to digital data by A/D convertor (71) for control unit (18) and finally can be seen on monitor (5).

Oscillometric measurement of NIBP blood pressure of the patient is done by using sphygmomanometers cuff (14) in FIG. 3. Cuff (14) sends blood pressure signals to processing circuit NIBP signal (76). This circuit changes various signals to a final signal that is the base for measurement. The digital signal form A/D convertor (79) is sent to control unit (18) and finally becomes ostensible and is shown on monitor (5). Blood pressure measurement is controlled by monitor (5). Also motor (73) and valve (74) are in touch with control unit (18) by motor drive (77) and valve drive circuit (78). Motor (73) produces pneumatic power that is transferred to windpipe (72) when valve (74) is opened, and cause to bloat cuff (14).

Signals of body temperature of the patient are sent from temperature sensor ((12) in FIG. 4) to temperature detect circuit (80) and then after digitalizing by A/D convertor (81) will be entered to control unit (18) and finally temperature will be shown on monitor (5).

1- An automated heart-abdomen external masseur package comprising: a plate placed under a patient's back, wherein two arms (first and second arms) extend from each side of said plate around chest and abdomen of said patient, wherein said first and second arms connect on top of said patient's chest and abdomen area housing: a chest masseur, a display touch-screen monitor, an abdomen masseur package having an abdomen massage pad, a central control unit comprising an on/off switch, batteries, an external power inlet, first and second ultrasonic sensors, a sphygmomanometer cuff, a defibrillator, multiple ECG lids, body temperature sensors, pulse oximetry; wherein data received when said external masseur is turned on is transferred to said central processing unit and a processor therefore further work of chest and abdomen motors is adjusted and controlled as needed.

2- The external masseur package of claim 1, wherein said plate and said first and second arms are made of non-flexible carbon fiber material.

3- The external masseur package of claim 2, wherein vital symptoms are observed and displayed on said monitor.

4- The external masseur package of claim 3, wherein after cardiac arrest said plate is placed under said patient's back and shoulders, while said first and second arms are wrapped around said patient's body covering said chest and abdomen; wherein said chest masseur is placed on a middle section of said chest; wherein said plate comprises an adjustable width accommodating different patient size.

5- The external masseur package of claim 4, wherein said plate comprises sliding joints one and two, creating said adjustable width.

6- The external masseur package of claim 5, wherein said chest masseur comprises a frame, said chest motor, a chest driver gear, a motor shaft and massage shaft having teeth or indentations; wherein a combination of said chest driver gear and said teeth form a set of rack and pinion mechanism, therefore rotational movement of said chest driver gear causes said massage shaft to vertically move up and down simulating CPR movement, therefore vertically moving said chest masseur up and down; wherein said first and second ultrasonic sensors measure a distance between said patient's chest and said chest masseur, therefore adjusting said vertical movement of said chest masseur, and wherein said chest masseur moves down only equal to said distance.

7- The external masseur package of claim 6, wherein continuous movement of said chest masseur compresses and decompresses said patient's chest at least 100 times per minute, with a minimum 2 inch replacement.

8- The external masseur package of claim 7, wherein said abdomen masseur is placed on said patient's chest and stomach on inferior vena cava and abdominal aorta.

9- The external masseur package of claim 8, wherein said central control unit compares on-location and continuous cardiogram of said patient with pre-defined/pre-recorded, normal and healthy cardiogram stored in a memory of said external masseur package, therefore calculating a correct amount of electrical shock needed for said defibrillator; wherein said correct amount is displayed on said display monitor; wherein continuous measurement of ECG waves and said cardiogram and data analysis will define further automatic chest and abdomen messaging via said chest and abdomen masseurs respectively.

10- The external masseur package of claim 9, wherein said abdomen masseur comprises two adjustable abdominal arms, adjusting a distance between said patient's chest and said abdomen, till said abdomen massage pad is placed below said patient's chest.

11- The external masseur package of claim 10, wherein said abdomen massage pad vertically moves up and down as the same rhythm as said chest masseur (same numbers, same depth and same speed), wherein a pressure will be applied on said inferior vena cava and abdominal aorta, compressing said patient's abdomen at least half an inch.
